(19) United States
(12) Patent Application Publication
(10) Pub. No.: US 2026/0315713 A1
(43) Pub. Date: Oct. 15, 2026

(54) MEDICAMENT DELIVERY SYSTEM WITH AUDIBLE VISUAL FLOW INDICATOR AND METHODS FOR THE USE AND ASSEMBLY THEREOF

(71) Applicant: Trudell Medical International Inc., London (CA)

(72) Inventors: Matthew Gravett, London (CA); Emmanuel Marbina, London (CA)

(73) Assignee: Trudell Medical International Inc., London (CA)

(21) Appl. No.: 19/571,908

(22) Filed: Mar. 19, 2026

Related U.S. Application Data
(60) Provisional application No. 63/774,976, filed on Mar. 20, 2025.

(51) Int. Cl. A61M 15/00

(52) U.S. Cl. CPC A61M 15/0086; A61M 15/0065; A61M 2205/183; A61M 2205/43; A61M 2205/502

(57) ABSTRACT

A valved holding chamber assembly includes a holding chamber having an input end configured to receive a pressurized metered dose inhaler and an output end. An inhalation valve disposed at the output end defines a first inhalation flow path between the input end and a user interface. A flow channel bypasses the inhalation valve and defines a second inhalation flow path with an audible flow indicator actuatable in response to a flow rate during inhalation. A visual flow indicator defines a third inhalation flow path including an ambient air inlet in flow communication with an ambient environment. The visual flow indicator is moveable from an at-rest position to an inhalation position, closing the third inhalation flow path when in the inhalation position. The visual flow indicator may include a hammer that strikes an anvil when moving to the inhalation position, emitting an audible signal.

### RELATED APPLICATION

This application claims the benefit of U.S. Provisional Application No. 63/774,976, filed on Mar. 20, 2025, pending, the entirety of which is herein incorporated by reference.

### TECHNICAL FIELD

The present invention relates to a medicament delivery system, for example and without limitation a valved holding chamber (VHC), having an audible visual flow indicator, and to systems and methods for the use and assembly thereof.

### BACKGROUND

The use of an aerosol medication delivery apparatus and system to administer medication in aerosol form to a patient's lungs by inhalation (hereinafter “aerosol delivery system(s)”) is well known in the art. As used herein, the term “substance” includes, but is not limited to, any substance that has a therapeutic benefit, including, without limitation, any medication; the terms “patient” and “user” include humans and animals; and the term “aerosol delivery system(s)” includes pressurized metered-dose inhalers (pMDIs), pMDI add-on devices, such as valved holding chambers, devices including a chamber housing and integrated actuator suited for a pMDI canister, nebulizers and dry powder inhalers. Examples of such aerosol delivery systems are disclosed in U.S. Pat. Nos. 4,627,432, 5,582,162, 5,740,793, 5,816,240, 6,026,807, 6,039,042, 6,116,239, 6,293,279, 6,345,617, and 6,435,177, the entire contents of each of which are incorporated herein by reference.

Conventional pMDIs typically have two components: 1) a canister component in which the medication particles and a propellant are stored under pressure in a suspension or solution form; and 2) a receptacle component, often referred to as an actuator boot, used to hold and actuate the canister and typically configured with a mouthpiece. The canister component typically includes a valved outlet from which the contents of the canister can be discharged. A substance is dispensed from the pMDI by applying a force on the canister component to push it into the receptacle component thereby opening the valved outlet and causing the medication particles to be conveyed from the valved outlet through the receptacle component and discharged from an outlet of the receptacle component. Air is drawn through the pMDI during inhalation, with the pMDI defining an airflow resistance. Upon discharge from the canister, the substance particles are “atomized” to form an aerosol.

PMDI holding chambers typically include a chamber housing with an input end and an output end. The mouthpiece portion of the pMDI receptacle is received in a backpiece located at the input end of the chamber housing. An example of such a backpiece is disclosed in U.S. Pat. No. 5,848,588, the entire contents of which are incorporated herein by reference. The output end of the chamber housing may include an inhalation valve or a containment baffle, or both, and a user interface, such as an adapter, a mouthpiece and/or a mask. The user interface may be coupled to the output end of the chamber housing or integrally molded with, and define in part, the output end of the chamber housing. Some holding chambers include an integrated receptacle for a pMDI canister thereby eliminating the need for a backpiece or other equivalent structure used to receive and hold the mouthpiece portion of a pMDI.

Pressurized metered dose inhalers (pMDIs) come in wide variety shapes and sizes, such that different pMDIs may have different airflow resistance characteristics. As the user inhales through a pMDI, the user creates a pressure drop between their mouth and atmospheric/ambient air in the user environment surrounding the pMDI. Depending on the resistance of the inhaler, air will flow through the pMDI at a given rate. The relationship between flow pathway resistance (R), pressure (P) and flow rate (Q) is defined by:

R=PQ

Given a constant pressure drop across any particular pMDI, the air flow rate through the pMDI is dictated by the pMDI's resistance; as the pMDI resistance increases, flow decreases, and vice versa.

Valved holding chambers may incorporate an audible flow indicator, often called a whistle, or harmonica, which may indicate an excessive inhalation flow rate during aerosol drug delivery. Excessive inhalation flow rates may lead to impaction of aerosolized drug particles in the upper airway of the user, thereby diminishing the amount of drug that may be delivered to the lungs. The whistles may be located in the backpiece of the holding chamber. If excessive inhalation flow rate occurs, then there is sufficient airflow passing through the whistle to actuate the whistle, for example by resonation, and make a sound, thereby alerting the user to alter their inhalation technique. Alternatively, some valved holding chambers may have a flow indicator, such as a whistle, built into the mouthpiece that is used for “positive” feedback, meaning that the whistle sounds when the user had achieved a minimum flow rate, as opposed to the “negative” feedback for excessive inhalation flow rate. In both embodiments, the whistle communicates directly with the ambient environment, meaning the flow through the flow indicator comes from the ambient environment surrounding the holding chamber. In this way, the flow indicator, e.g., whistle, and pMDI connect the chamber body to the ambient environment, or air, in a “parallel” arrangement, as shown schematically in FIG. 1. In this arrangement, the flow rate is dependent on the pressure and resistance of both the whistle and the pMDI:

Qinhalation=QpMDI+QWhistle=P/RpMDI+P/PWhistle

In order for the airflow rate through the whistle to be consistent at a given pressure, without regard to the type of pMDI being used, the expression would be:

P/RpMDI1+P/RWhistle=P/RpMDI2+P/RWhistle

Given that the pMDIs have differing resistances, however, the parallel arrangement does not allow for the whistle to trigger at a consistent flowrate because R_{pMD/1}≠R_{pMD/2}.

Many aerosol delivery systems may also lack a visual indication to alert a caregiver when a patient is inhaling, for example below the excessive inhalation flow rate threshold. In the case of a pMDI used in conjunction with a holding chamber, for example, it may be helpful for a caregiver to know if the patient is inhaling at a rate sufficient to open the inhalation valve to allow the aerosolized medication to exit the holding chamber, and/or to confirm that the device is being used properly. It may also be helpful to know when the patient is inhaling, has stopped inhaling and/or is exhaling, in order to coordinate the actuation of the pMDI with inhalation. In some situations, it may be difficult to see the visual flow indicator, for example when a user, or caregiver is administering medication at night in a darkened room, or if a patient or caregiver has poor vision and has difficulty seeing the visual indicator even with adequate lighting.

### SUMMARY

In one aspect, one embodiment of a valved holding chamber assembly includes a holding chamber adapted to contain a substance and having an input end adapted to receive a pressurized metered dose inhaler and an output end. An inhalation valve is disposed at the output end of the holding chamber and is moveable to an open position in response to an inhalation flow through the holding chamber and the inhalation valve. A user interface is coupled to the output end of the holding chamber in flow communication with the holding chamber when the inhalation valve is in the open position, wherein the inhalation valve defines a first inhalation flow path between the input end and the user interface. A flow channel includes an inlet in flow communication with the holding chamber between the input end and the output end and an outlet in flow communication with the user interface downstream of the inhalation valve, wherein the flow channel bypasses the inhalation valve, and wherein the flow channel defines a second inhalation flow path between the input end and the user interface. A flow indicator is positioned in the flow channel, wherein the flow indicator is actuatable in response to a flow rate in the flow channel during inhalation. In one embodiment, the flow indicator is configured as a whistle. In this embodiment, the flow channel is in flow communication with the holding chamber and the user interface, but is not in direct flow communication with the ambient environment.

In one embodiment, a second flow channel includes a second inlet in flow communication with the holding chamber between the input end and output end and a second outlet in flow communication with the user interface downstream of the inhalation valve. The second flow channel defines a third inhalation flow path between the input end and the user interface separate from the first and second inhalation flow paths. In this embodiment, the second flow channel is in flow communication with the holding chamber and the user interface, but is not in direct flow communication with the ambient environment. A second flow indicator may be positioned in the second flow channel, wherein the second flow indicator is actuatable in response to a second flow rate in the second flow channel during inhalation. In one embodiment, the inhalation valve has a first flow resistance, the first flow indicator has a second flow resistance, and the second flow indicator has a third flow resistance, wherein the second flow resistance is greater than the first flow resistance, and the first flow resistance is greater than the third flow resistance.

In another aspect, one embodiment of a valved holding chamber assembly includes a first flow path between an input end and a user interface, wherein the input end is configured for coupling to a medicament delivery device and wherein the first flow path includes an inhalation valve having a first flow resistance. A second flow path is defined between the input end and the user interface, wherein the second flow path bypasses the inhalation valve, wherein the second flow path includes a flow indicator having a second flow resistance, and wherein the second flow resistance is greater than the first flow resistance. In one embodiment, the flow indicator is configured as a whistle. In one embodiment, a third flow path is defined between the input end and the user interface, wherein the third flow path is separate from the first and second flow paths, wherein the third flow path has a second flow indicator having a third flow resistance, and wherein the first flow resistance is greater than the third flow resistance. Neither the second nor the third flow paths are in direct flow communication with the ambient environment. In another aspect, one embodiment of a medicament delivery system includes a housing defining a flow path, wherein the housing is configured with an anvil. A flow indicator is disposed across at least a portion of the flow path, wherein the flow indicator is moveable in response to an inhalation flow in the flow path from an at-rest position to an inhalation position. The flow indicator includes a hammer moveable from an at-rest position to an impact position as the flow indicator is moved in response to the inhalation flow from the at-rest position of the flow indicator to the inhalation position. The hammer impacts the anvil in the impact position and emits an audible signal.

In yet another aspect, one embodiment of a method of delivering a medicament includes inhaling through a patient interface portion of a housing, moving at least a portion of a flow indicator in a flow path from an at rest position to an inhalation position in response to the inhaling, striking an anvil with a hammer on the flow indicator as the flow indicator is moved to the inhalation position, and emitting an audible signal indicating that the flow indicator is in the inhalation position.

In another aspect, one embodiment of a valved holding chamber assembly includes a holding chamber having an input end configured to receive a pressurized metered dose inhaler and an output end. An inhalation valve is disposed at the output end of the holding chamber and is moveable to an open position in response to an inhalation flow through the holding chamber, wherein the inhalation valve defines a first inhalation flow path between the input end and a user interface. A flow channel has an inlet in flow communication with the holding chamber between the input end and the output end and an outlet in flow communication with the user interface downstream of the inhalation valve, wherein the flow channel bypasses the inhalation valve and defines a second inhalation flow path between the input end and the user interface. An audible flow indicator is positioned in the flow channel, wherein the audible flow indicator is actuatable in response to a flow rate in the flow channel during inhalation. A visual flow indicator defines a third inhalation flow path, wherein the third inhalation flow path includes an ambient air inlet in flow communication with an ambient environment. The visual flow indicator is moveable from an at-rest position to an inhalation position, wherein the visual flow indicator closes the third inhalation flow path when in the inhalation position.

In yet another aspect, one embodiment of a method of delivering a medicament includes inhaling through a user interface of a valved holding chamber, drawing air through a third flow path from an ambient environment and past a visual flow indicator at a first inhalation flow rate, moving the visual flow indicator from an at-rest position to an inhalation position in response to the first inhalation flow rate reaching a threshold flow rate, closing the third flow path with the visual flow indicator in the inhalation position, opening an inhalation valve in response to closing the third flow path, and drawing air through a first flow path including the inhalation valve and a second flow path including an audible flow indicator at a second inhalation flow rate greater than the first inhalation flow rate.

The various aspects and embodiments provide significant advantages over other medication delivery systems and methods. For example, and without limitation, in some embodiments, all of the external air from the ambient environment passes through the pMDI and into the chamber before flowing through any of the first, second and/or third flow paths to the user interface. In this way, the entire flow passes through the pMDI before splitting between the inhalation valve and the flow indicator, configured as a whistle in one embodiment, or before splitting between the inhalation valve and the second flow indicator. Since the inhalation valve and the flow indicator(s) each have a constant flow resistance, the flow indicator (e.g., whistle) may be configured to actuate at a specified flow rate, regardless of the pMDI being used with the valved holding chamber. In other words, the flow indicator(s) and inhalation valve are arranged in parallel, but the combination thereof is arranged in series with the pMDI, as shown in FIG. 2A and B.

In addition, actuation (e.g., movement) of the second flow indicator allows users and caregivers to visually detect when the user is inhaling when using an aerosol delivery system such that the user or caregiver may be alerted to possible defects and/or improper inhalation, including without limitation an improper seal being formed between the patient's face and the aerosol delivery system's interface, such as a mask.

The hammer and anvil interface provide an audible signal indicating that a proper inhalation flow has been produced, and may supplement and/or substitute for the visual indicia achieved by watching only the flow indicator. The combination of audible and visual indicator allows for the system and device to be used in multiple environments, including at nighttime without the need to turn on the lights.

In other embodiments, external air from the ambient environment passes through the pMDI and into the chamber before flowing through any of the first and/or second flow paths to the user interface, while external air from the ambient environment passes through an inlet directly into the third flow path to the suer interface.

The foregoing paragraphs have been provided by way of general introduction, and are not intended to limit the scope of the following claims. The various preferred embodiments, together with further advantages, will be best understood by reference to the following detailed description taken in conjunction with the accompanying drawings.

### BRIEF DESCRIPTION OF THE DRAWINGS

FIG. 1 is schematic representation of a prior art valve holding chamber.

FIG. 2A is a schematic representation of a first embodiment of a valved holding chamber.

FIG. 2B is a schematic representation of a second embodiment of a valved holding chamber.

FIG. 2C is a schematic representation of a third embodiment of a valved holding chamber.

FIG. 3A is a side cross-sectional view of a first embodiment of a valved holding chamber.

FIG. 3B is an enlarged partial cross-sectional view of the valved holding chamber taken along line 3B in FIG. 3A.

FIG. 4 is a perspective view of a second embodiment of a valved holding chamber.

FIG. 5 is a top view of the valved holding chamber shown in FIG. 4.

FIG. 6 is a side view of the valved holding chamber shown in FIG. 4.

FIG. 7 is an end view of the valved holding chamber shown in FIG. 4.

FIG. 8 is a cross-sectional view of the valved holding chamber shown in FIG. 4 taken along line 8-8.

FIG. 9 is a graph showing the resistance pressure v. flow rate for various exemplary pMDI devices.

FIG. 10 is a cross-sectional view of one embodiment of a valved holding chamber with the various flow paths illustrated.

FIGS. 11A-D illustrate various whistle embodiments.

FIG. 12 is a partial, cross-sectional view of one embodiment of a valved holding chamber.

FIG. 13A and B are partial cross-sectional view of one embodiment of a valved holding chamber with acoustical openings and valves.

FIG. 14 is an end view of one embodiment of the valved holding chamber showing the acoustical openings.

FIG. 15 is a perspective view of a visual flow indicator configured with a hammer.

FIG. 16 is partial, perspective view of the visual flow indicator in FIG. 15 arranged in a medicament delivery device.

FIG. 17 is a side schematic view of a visual flow indicator with first and second hammers and first and second anvils.

FIG. 18 is a cross-sectional view of a valved holding chamber during initial inhalation where a second flow path (FP2) and a third flow path (FP3) are active.

FIG. 19 is a cross-sectional view of a valved holding chamber during full inhalation where the visual flow indicator flap has closed the third flow path (FP3), and all inhaled air is drawn through a first flow path (FP1) and the second flow path (FP2).

FIG. 20 is a perspective close-up view of an alternate embodiment of the audible visual flow indicator with the third flow path (FP3) inlet from ambient and a one-sided hammer.

FIG. 21 is a side cross-sectional close-up view of an alternate embodiment of the audible visual flow indicator with the third flow path (FP3) inlet from ambient and a one-sided hammer.

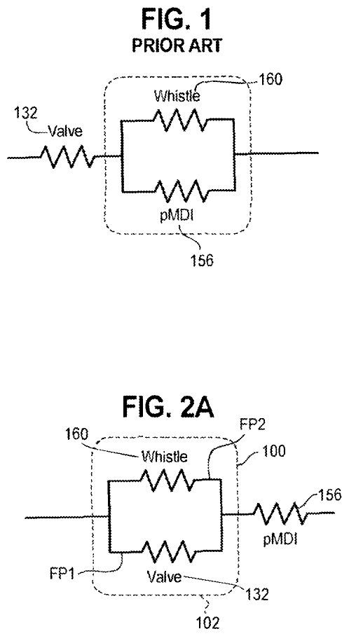

### DETAILED DESCRIPTION OF THE DRAWINGS AND PRESENTLY PREFERRED EMBODIMENTS

It should be understood that the term “plurality,” as used herein, means two or more. The terms “longitudinal” as used herein means of or relating to length or the longitudinal direction 2, for example between the opposite ends of the holding chamber or flow channel. The terms “lateral” and “transverse” as used herein, means situated on, directed toward or running from side to side, and refers to a lateral direction 4 orthogonal to the longitudinal direction. The term “direction” corresponds to an axis or line, rather than a vector. The term “coupled” means connected to or engaged with whether directly or indirectly, for example with an intervening member, and does not require the engagement to be fixed or permanent, although it may be fixed or permanent (or integral), and includes both mechanical and electrical connection. The terms “first,” “second,” and so on, as used herein are not meant to be assigned to a particular component so designated, but rather are simply referring to such components in the numerical order as addressed, meaning that a component designated as “first” may later be a “second” such component, depending on the order in which it is referred. For example, a “first” inlet may be later referred to as a “second” inlet depending on the order in which they are referred. It should also be understood that designation of “first” and “second” does not necessarily mean that the two features, components or values so designated are different, meaning for example a first inlet may be the same as a second inlet, with each simply being applicable to separate but identical components. As used herein: the term “substance” includes, but is not limited to, any substance that has a therapeutic benefit, including, without limitation, any medication; the terms “user” and “patient” includes humans and animals; and the term “aerosol delivery devices or system(s)” includes pressurized metered-dose inhalers (pMDIs), pMDI add-on devices, such as holding chambers, devices including a chamber housing and integrated actuator suited for a pMDI canister, nebulizers and dry powder inhalers. The phrase “flow communication” refers to two or more components or features constructed and arranged to provide for a flow of fluid (gas or liquid) therebetween, meaning for example a fluid may flow between an inlet and a holding chamber that are in “flow communication.” Two or more components or features may be in “flow communication” when a component, such as a valve, is open, but are not in flow communication when the component, such as the valve, is closed.

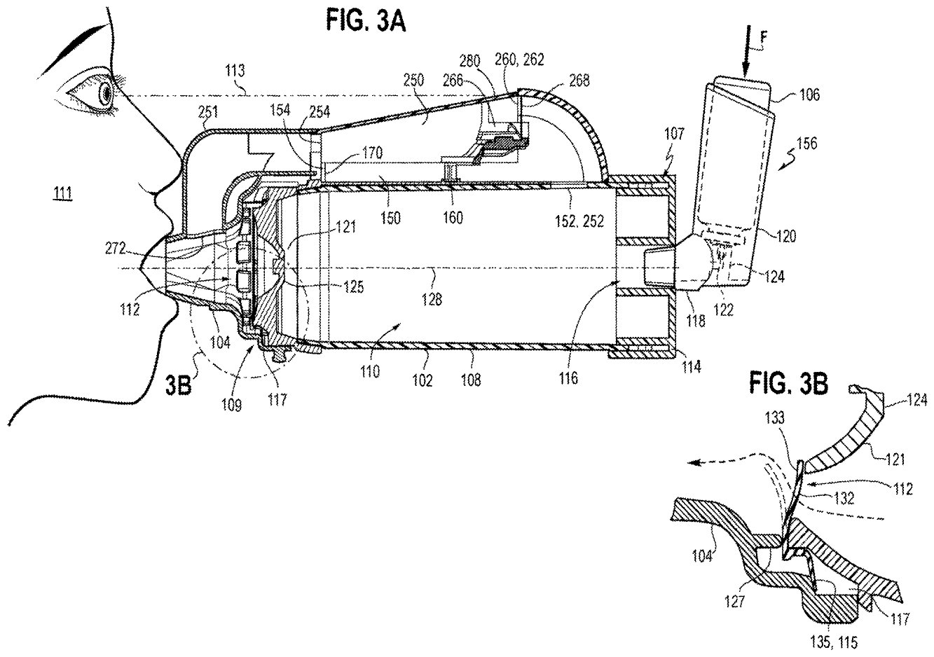

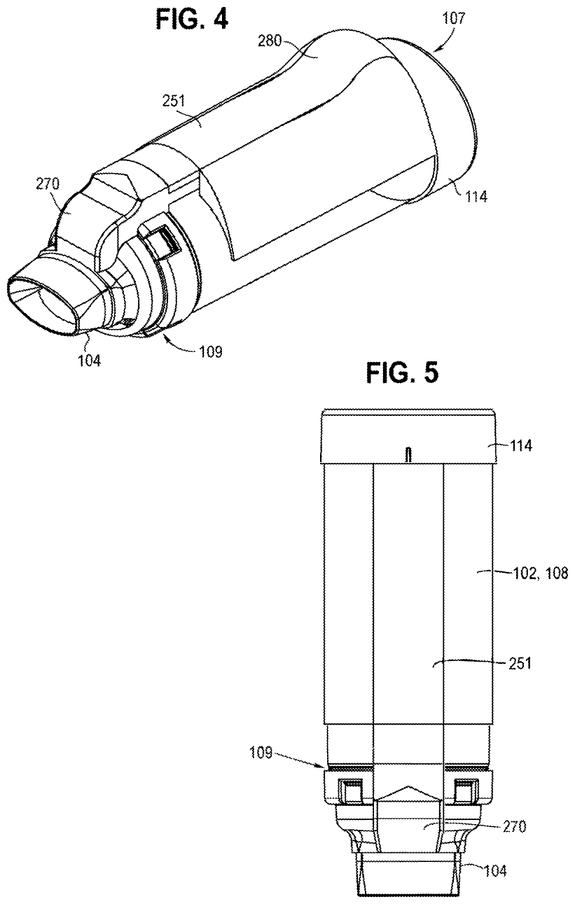

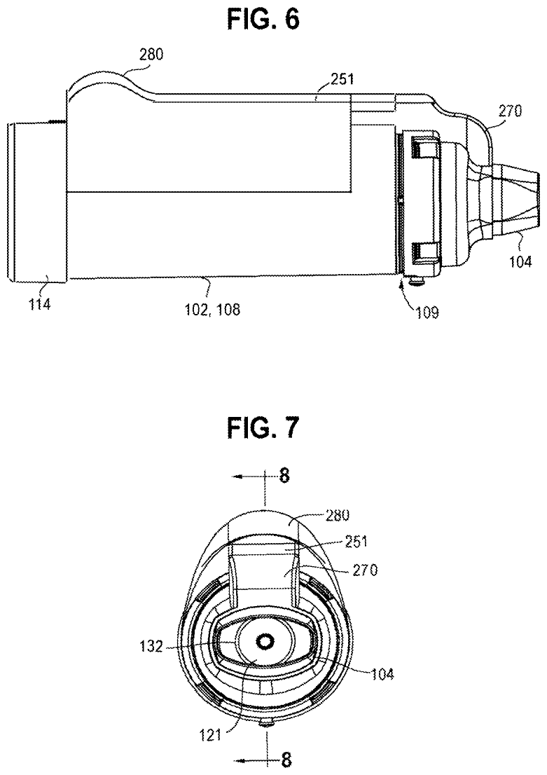

FIGS. 3A-8 show different embodiments of an aerosol delivery system 100. The system 100 includes a holding chamber 102 or conduit, a user interface 104, an inhalation valve 132 and a source of a substance, such as a pMDI canister 106, attached to a rear input end 107 of the holding chamber 102. The holding chamber 102 includes a chamber housing 108 that has a generally cylindrical shape with a generally circular cross-sectional shape that defines an interior volume 110 of space for receipt therein of aerosolized medication from the pMDI 156. A front output end 109 of the chamber housing includes an outlet 112, configured in one embodiment as an opening that is in fluid or flow communication with the interior volume 110 of space of the chamber housing 108. The user interface 104 may be configured as a mouthpiece, mask (nasal or oral/nasal), tube, or other suitable user interface, which is in fluid communication with the outlet opening 112. The inhalation valve 132 may be disposed over the opening to provide for one-way flow through the opening during inhalation but prevents back flow into the interior volume 110 during exhalation. The inhalation valve may be configured as any type of valve that permits one-way flow, including without limitation a flap valve, a duck-bill valve, an annular donut valve (i.e., valve having a central opening with a peripheral sealing edge), a center post valve, and/or a slit petal valve.

An exhalation valve 115 may be provided to allow for one-way flow to the ambient environment surrounding the system 100 during exhalation, but prevents air entrainment through an exhalation opening 117 during inhalation. The exhalation valve 115 may be configured as any type of valve that permits one-way flow, including without limitation a flap valve, a duck-bill valve, or an annular donut valve (i.e., valve having a central opening with a peripheral sealing edge), a center post valve, and/or a slit petal valve, and may be integrally formed with the inhalation valve as a single valve component.

The input end 107 of the chamber housing 108 is attached to, or includes, a detachable and flexible backpiece 114, or adapter, having an inlet 116, configured in one embodiment as an opening suited to receive the mouthpiece portion 118 of the pMDI receptacle 120, otherwise referred to as an actuator boot, that houses the pMDI canister 106. An exemplary pMDI 156 includes the canister 106 coupled to the receptacle 120. The canister 106 includes a valve stem 122 disposed in a well 124 in the bottom of the receptacle 120. The inlet 116 is in fluid communication with the interior volume 110. Examples of possible pMDI adapters and canisters to be used in conjunction with the holding chamber 102 are also described in U.S. Pat. Nos. 5,012,803, 5,012,804, 5,848,588 and 6,293,279, the entire contents of each of which is incorporated herein by reference. It should be understood that other aerosol delivery systems may have various one or more interior volumes fillable with an aerosolized medicament, with one or more outlets and one or more inlets communicating with the interior volume, including devices having a chamber housing and integrated actuator suited for a pMDI canister, nebulizers, dry powder inhalers and other such devices. The holding chamber 102 extends in the longitudinal direction 2, with the inlet 116 and outlet 112 being longitudinally spaced.

In one embodiment, a force F may be applied to the canister, thereby moving the valve stem 122 of the pMDI canister to discharge a predetermined dosage of medicament from the discharge end, e.g., mouthpiece portion 118, of the pMDI receptacle in aerosol form from the input end 107 into the interior volume 110 of the chamber housing 108. The aerosol medication particles within the interior volume 110 and chamber housing 108 are thereafter withdrawn through the outlet opening 112 at the output end 109 by having the user/patient 111 inhale through the interface 104.

The pMDI canister 106 contains a substance, preferably a medication suspension or solution under pressure. For example, the substance dispensed may be an HFA propelled medication suspension or solution formulation. Other medicaments, or medications, and propellants, such as CFC may also be used. It should be pointed out that while the described embodiments regard an aerosol delivery system for the delivery of an aerosolized medication from a pMDI, other aerosol delivery systems are contemplated that can be used within the spirit of the present invention. For example, it is contemplated that the flow indicator(s) may be incorporated with an aerosol delivery system such as existing ventilator systems, dry powder inhalers and nebulizers, in a manner similar to that described below. Examples of nebulizers that can be adapted to include such an indicator are disclosed in U.S. Pat. Nos. 5,823,179 and 6,044,841, the entire contents of which are incorporated herein by reference.

The present embodiments are not limited to the treatment of human patients. For example, it is contemplated that valved holding chamber may be configured with a use interface (e.g., mask) for administering medication to animals, including for example and without limitation equines, cats, dogs, etc.

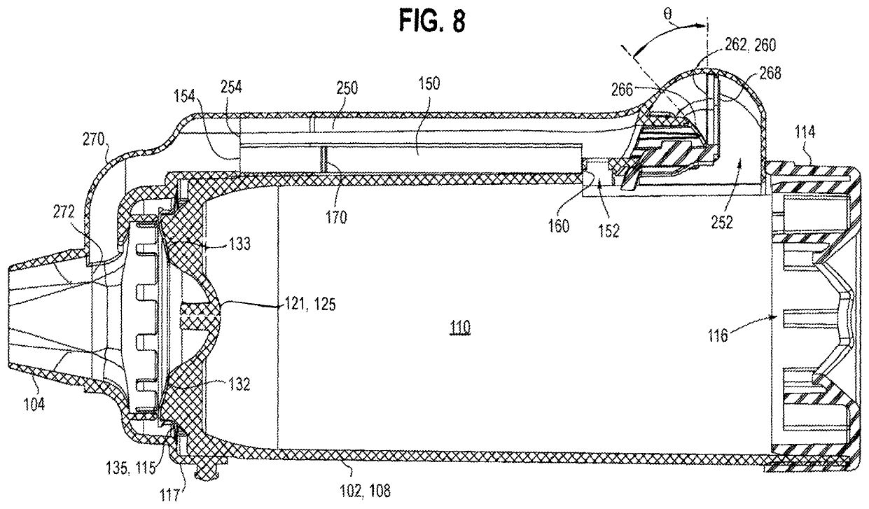

Referring to FIGS. 3A, 3B and 8, the output end 109 may include a containment baffle 121 positioned so as to partially block the opening 112. The baffle 121 reduces the velocity or flow rate or both of the aerosol medication particles flowing along the axis 128 of the chamber housing 108. A circular dome portion 125 of the baffle is aligned with the central axis 128 of the chamber housing 108 and is directly in line with the opening 112. Aerosol medication particles that have a flow path away from the central axis 128 tend to have a velocity that is lower than that of particles near to the axis 128. The baffle 121 may be configured with the dome portion 125, which reduces the forward, on-axis velocity and simultaneously acts as an impaction surface for on-axis projectile aerosol medication particles and so protects the valve 132. The dome portion 125 protrudes toward the input end 107. At the same time, the dome portion 125 allows slower moving aerosol medication particles to migrate towards the sides 130 of the chamber housing 108. It should be understood that the dome portion can alternatively be formed with a flat surface facing the input end, or a curved surface, for example a convex or concave surface.

As shown in FIGS. 3A, 3B, and 8, an annular valve 132 includes an inner inhalation valve portion 133 and an outer exhalation valve portion 135. The valve 132 is seated on the output end 109 of the holding chamber. The user interface 104, shown as a mouthpiece, includes retaining members 127 that clamp the valve 132 between the retaining members and the output end of the holding chamber. The valve may be made of a soft plastic, such as silicone or a thermoplastic elastomer.

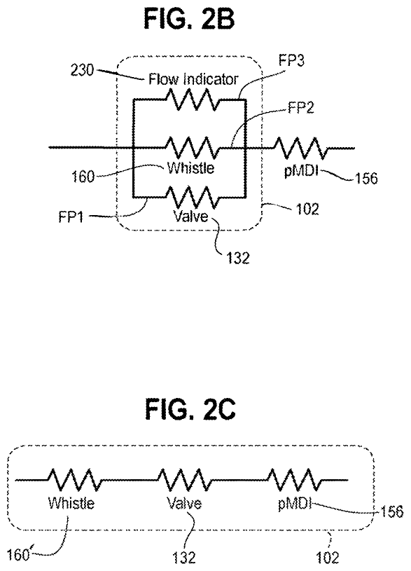

Referring to FIGS. 2A, 2B, 3 and 8, the aerosol delivery system 100, otherwise referred to as an aerosol delivery device, is configured with one or more flow channels 150, 250, which are preferably arranged on a top of the holding chamber such that the flow channels 150, 250 are visible to the user when using the device. It should be understood that the flow channels may be arranged, and extend radially from the holding chamber, at any location, including a bottom or side thereof. The flow channels may also be embedded radially inwardly from an outward surface of the holding chamber. In one embodiment, the flow channels are defined by an auxiliary housing 251 connected to the holding chamber 102. The auxiliary housing 251 may be integrally formed with the holding chamber, or formed separately and coupled thereto with tabs, fasteners, adhesives and the like.

The inhalation valve 132 is disposed at the output end 109 of the holding chamber and is moveable to an open position in response to an inhalation flow through the holding chamber 102. The user interface 104 is coupled to the output end 109 of the holding chamber in flow communication with the holding chamber when the inhalation valve is in the open position. In this way, the inhalation valve 132 defines a first inhalation flow path (FP1) between the input end, and the pMDI in particular, and the user interface, with reference to FIGS. 2A and 2B.

The auxiliary housing 251 defines a first flow channel 150 having an inlet 152 in flow communication with the holding chamber 102 between the input end 107 and the output end 109 and an outlet 154 in flow communication with the user interface 104 downstream of the inhalation valve 132, for example with the mouthpiece. The flow channel 150 is not in direct flow communication with the ambient environment, but rather is closed off or isolated from the ambient environment as the flow channel is in direct flow communication between the holding chamber 102 and the user interface 104. The flow channel 150 bypasses the inhalation valve 132 and defines a second inhalation flow path (FP2) between the input end, and in particular the pMDI 156, and the user interface 104. A flow indicator 160 is positioned in the flow channel 150. The flow indicator 160 is actuatable in response to a predetermined flow rate in the flow channel 150 during inhalation. In one embodiment, the flow indicator 160 is configured as an audible flow indicator, which may be a whistle. In one embodiment, the flow indicator 160′ may include a flexible strand disposed in the user interface 104, for example in the mouthpiece, in series with the inhalation valve 132 and pMDI 156, as shown in FIG. 2C. The strand may snap and wave like a flag during excessive inhalation.

Referring to FIGS. 2A, 2B, 3 and 8, the flow indicator 160 is actuatable when the flow rate in the flow channel 150 is greater than a threshold rate. The threshold flow rate in the flow channel 150 may include from 3 lpm-18 lpm (including for example 6 lpm), such that the flow from the flow channel 150 and the flow passing through inhalation valve 132 in combination are between 10 and 100 lpm, including for example 60 lpm, so as to provide indicia to the user that the inhalation rate is excessive.

In another embodiment, the inhalation valve incorporates, or is integrally formed with the flow indicator. For example, the inhalation valve may be configured as a duckbill valve having portions that flap excessively in response to excessive inhalation. Such embodiments may include a thin reed, formed from an elastic material, within the inhalation valve. The thin reed would flap excessively in response to excessive inhalation. This flapping would generate vibrations that can be felt by the user, and/or audible sound waves, as to alert the user of excessive inspiratory flow rate. In this embodiment, the valve 132 and flow indicator 160′ are in series, albeit integrally formed.

In one embodiment, an inhalation valve 170 may be disposed in the flow channel 150 defining the second inhalation flow path FP2, such that flow through the first flow channel 150 is one-way or unidirectional. The valve 170 may prevent moisture and residual drug contained in the mouthpiece area and exhaled breath from entering the flow channel where it could deposit and contaminate the flow channel. It also prevents exhaled air from flowing back through the flow channel into the chamber where it could cause remaining suspended aerosol drug that has not yet been inhaled to be pushed out of the chamber. In this aspect, it operates in the same manner that the inhalation valve 132 does when the user begins to exhale by ensuring air does not back-flow into the chamber and thereby affect the drug delivery. In one embodiment, the flow channel 150 may be configured with an exhalation flow indicator, for example a whistle, which is actuated in response to an excessive exhalation flow rate after drug inhalation, which may reduce the amount of drug delivered to the lungs. In this embodiment, the flow channel is not configured with an inhalation valve. In one embodiment, the flow indicator 160 may operate in response to both an inhalation and exhalation flow.

In one embodiment, two inhalation flow indicators, preferably one being a whistle 160 and the other being a visual indicator 260, may be positioned in series within a single flow channel 150. In this arrangement, as the user inhales, the visual indicator 260 moves forward in response to inhalation and reaches a maximum position as described with respect to other embodiments. However, the visual flow indicator 260 does not close off the channel 150 to flow, but rather allows flow to pass by and continue on through the channel and through the whistle 160. Alternatively, the whistle 160 may be positioned upstream of the visual indicator 260. In either position, when inhalation stops and exhalation begins, the visual indicator 260 returns to an at-rest position and closes off the flow channel 150 and in doing so, acts as an exhalation valve closing off the flow channel 150 to exhalation flow. In this embodiment the visual indicator 260 advantageously functions as both a visual indicator and an inhalation valve eliminating the need for the separate inhalation valve.

In one embodiment, the auxiliary housing 251 also defines a second flow channel 250 having a second inlet 252 in flow communication with the holding chamber 102 between the input end 107 and the output end 109 and a second outlet 254 in flow communication with the user interface 104 downstream of the inhalation valve 132. The first and second inlets may be defined by the same opening, as shown in FIG. 3, or different openings as shown in FIG. 8. Likewise, the first and second outlets may be defined by the same opening, or different openings. As shown in the embodiment of FIGS. 3A and 8, the outlets 154, 254 open into a passageway 270 having an outlet 272 in direct flow communication with the user interface 104. In one embodiment, the passageway 270 has a ninety-degree) (90° bend, or between a 30 and 60 degree bend, between the outlets 154, 254 and the outlet 272. In other embodiments, the passageway may have other orientations, or be straight, depending on the configuration and positioning of the user interface. The second flow channel 250 defines a third inhalation flow path (FP3) between the input end 107, and the pMDI 156 in particular, and the user interface 104 separate from the first and second inhalation flow paths FP1 and FP2. A second flow indicator 260 may be positioned in the second flow channel 250, wherein the second flow indicator is actuatable in response to a second flow rate in the second flow channel 250 during inhalation. In one embodiment, the second flow indicator 260 is configured as a visual flow indicator, defined by a moveable valve member 262. In one embodiment, the second flow indicator includes the valve member 262 moveable to a closed inhalation position, wherein the valve member engages or is seated on a valve seat 266 in response to the inhalation flow through the second channel. After the valve member engages the seat 266, the flow in the flow channel 250 is stopped. When the inhalation flow is stopped, the valve 262 returns to an at rest, or closed exhalation position, wherein the valve member engages or is seated on a valve seat 268. In the absence of any flow, or in response to an exhalation flow, the valve 262 is moved to the at rest position and prevents any exhalation flow from entering the holding chamber through the inlet 252. The valve member 262 is visible to the user and/or care giver through a viewing port 280, which may be defined by a transparent portion of the auxiliary housing 251.

The viewing port 280 may be slightly elevated above the rest of the auxiliary housing, such that the user 111 engaged with the user interface 104 has a line of sight 113 to the viewing port 280, and the visual flow indicator 260 disposed therein. It should be understood that the flow channel 250 and flow indicator 260 are optional, meaning the holding chamber may be configured with only one flow channel 150 and flow indicator 160. In the embodiments shown in FIGS. 3A and 8, the viewing port 280, and flow indicator 260 are located at a distal end of the holding chamber 102, or closer to the input end 107 than the output end 109, with the inlets 152, 252 also located closer to the input end 107 than the output end 109. By positioning the flow indicator at a distal location, the visibility of the flow indicator is improved and limits or prevents the user from having to cross their eyes. In other embodiments, the flow indicator may be located closer to the output end 109 than the input end 107.

With the channel inlet 152 positioned at the distal end of the chamber, the amount of medication or drug that may enter the channel 150 is minimized. When the pMDI 156 is actuated, a medication plume advances towards the proximal end of the chamber, impacts the baffle 121, or domed portion 125, and then disperses or distributes throughout the chamber back towards the distal end. Once the user inhales through the chamber, which normally takes place shortly before or after the pMDI is actuated, the volume of air in the chamber, and therefore the suspended aerosolized drug, begins to evacuate through the inhalation valve 132 at the proximal end, with external air being pulled into the distal end of the chamber via the pMDI 156.

Therefore, the channel 150, 250 intakes predominately new external air since the channel inlet 152, 252 is located at the distal end of the chamber, close to the pMDI 156. There may be some aerosolized medication that enters into the flow channel 150, 250, however the particle sizes are expected to be small considering most of the large particles would impact on the inside of the chamber closer to the proximal end/baffle 121. Also, due to timing of inhalation vs. pMDI actuation, the concentration of drug near the flow channel inlet 152, 252 is typically low.

Therefore, the low quantity of small particle size medication that passes through the channel 150, 250 is more likely to make it through the flow channel and to the user's lungs in any event. Reducing the amount of drug entering the channel 150, 250 may help avoid drug build-up in the channel 150, 250, which may adversely impact the function of the whistle 160 and flow indicator 260.

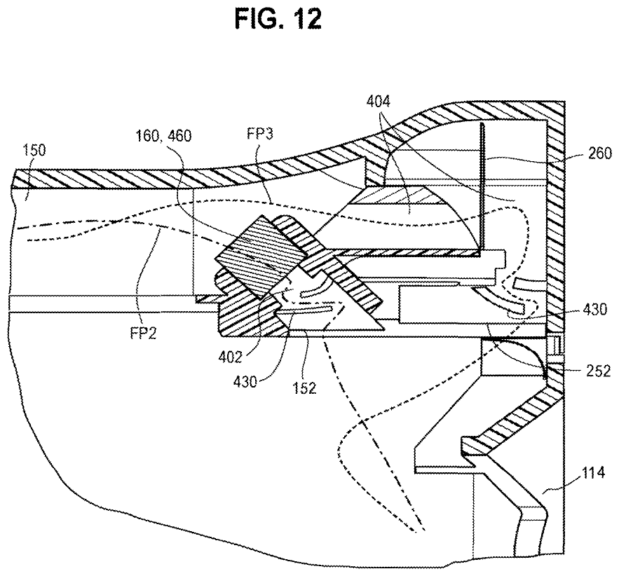

To further prevent any drug entering the flow channel 150, for example through channels 402, 404, or flow channel 250, a filter 420 may cover the inlet 152, 252. It should be understood that a single inlet 152 may feed both flow channels 402, 404. In one embodiment, a low flow resistance mesh filter 420 may be placed at the channel inlet 152, 252 to prevent drug from building up inside the channel 150, 250, 402, 404 and on the whistle 160, 460 and visual flow indicator 260. Such build-up my impact the performance of the whistle 160, 460 by affecting the airflow and resonance frequency of the reed(s) 462, or causing the visual flow indicator 260 to stick to any contacting surfaces. The filter 420 may be provided, or assembled, on an accessible area of the chamber for easier cleaning or replacement. The mesh filter 420 may be integrally molded with the holding chamber, or may be removable. The filter 420 may incorporate static properties to ensure the drug particles are attracted to and captured by the filter 420. In an alternative embodiment, shown in FIG. 12, a baffle 430 may be used instead of, or in combination with, the filter 420. The baffle 430 may be disposed at the inlet 152, 252 of each channel 150, 250, 402, 404, or a single baffle may be disposed at a single inlet feeding multiple channels 402, 404. The baffle 430 causes particles to impact on the baffle by creating a tortuous path through the flow path. The baffle 430 may be located within the chamber in area that is easy to clean.

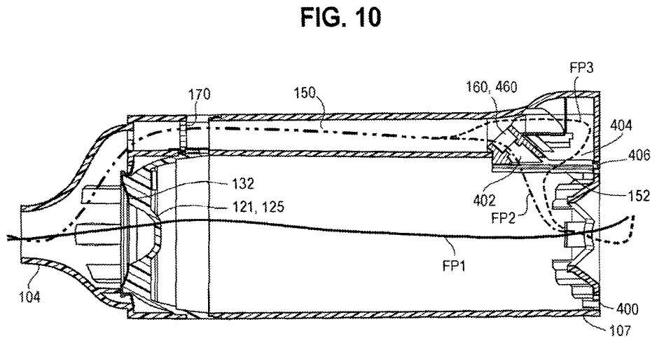

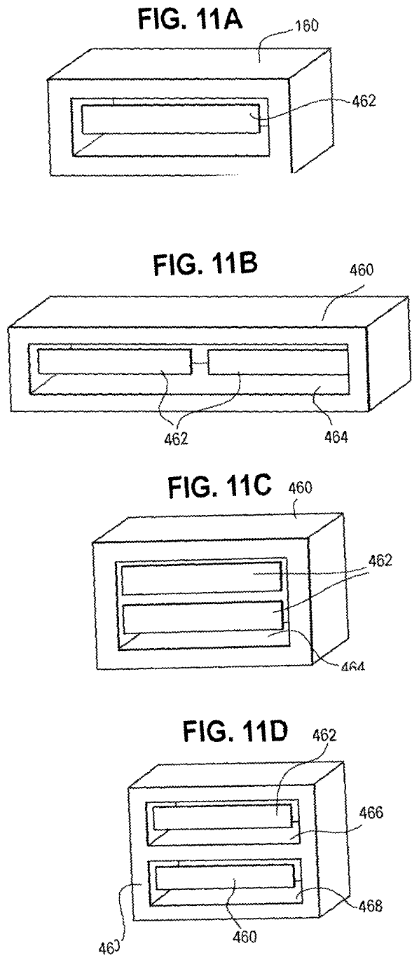

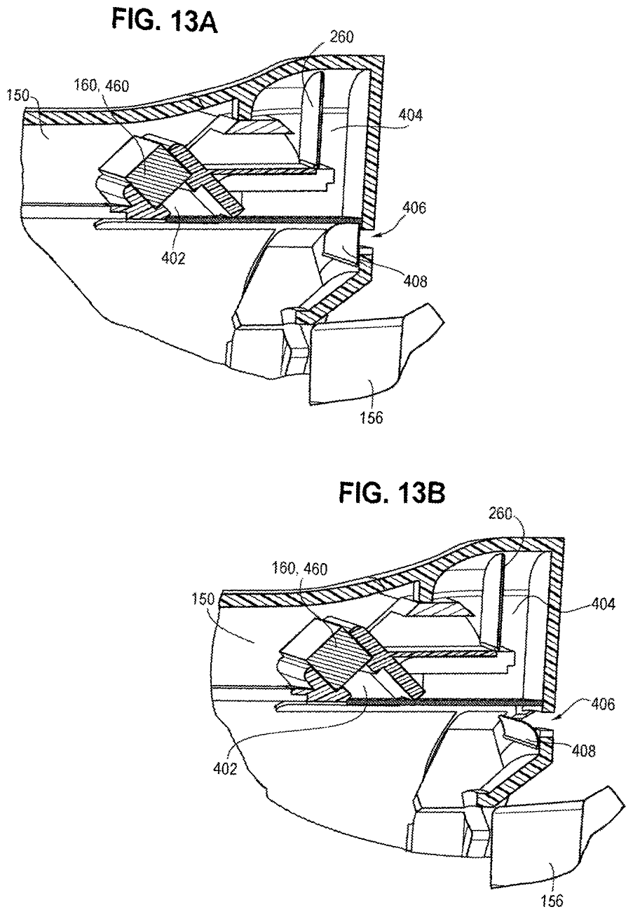

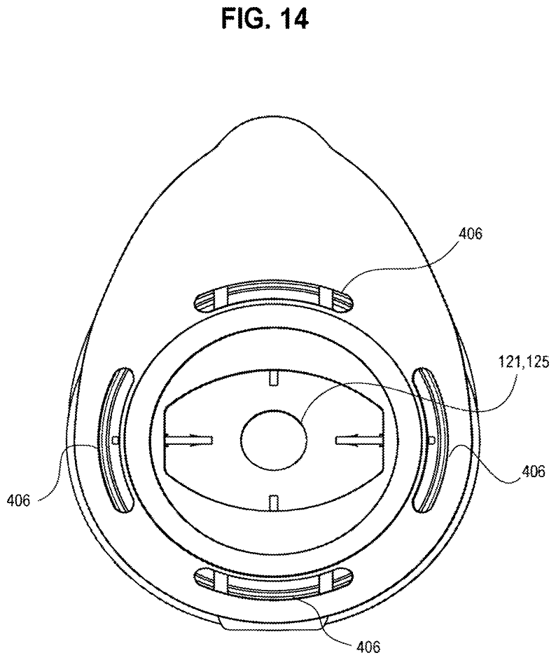

Referring to FIGS. 10, 11A-D, 13A and B and 14, a single flow channel 150 is provided, but with two flow paths FP2, FP3 having channels 402, 404 leading to the flow channel 150. An audible flow indicator 160, configured for example as a whistle, may be disposed in the channel 404 and flow path FP2. In order for the user or caregiver to hear the whistle, located in the flow channel 404, the sound may need to propagate through the channel 404, 150, into the chamber and through any gaps in the pMDI body and into the environment. In order to optimize the sound intensity of the whistle, one or more acoustical openings 406, otherwise referred to as sound vents or cutouts, may be formed in the adapter 114. The acoustical opening(s) 406 enhances sound propagation by eliminating the need for sound to propagate through the pMDI 156, i.e., flow channels formed therein, by providing the acoustical opening(s) 406 proximate the audible flow indicator 160, preferably located in the distal end of the chamber in one embodiment. The proximity reduces the distance the sound has to travel to reach the ambient environment and with fewer obstructions that may reduce the sound intensity. The audible flow indicator 160, e.g., whistle, may also be angled (i.e., having a vector intersecting the adapter) within the channel 404, as shown in FIG. 10, such that the maximum sound energy is directed towards the adapter and the sound vents to maximize the sound intensity to the ambient environment and the user. As it relates to flow, the acoustical opening(s) 406 may introduce a slight secondary flow path in addition to the air entering the chamber through the pMDI/adapter inlet. In use, this may have minimal impact including negligible effects on aerosolized drug delivery. Limited aerosolized medicament escapes from the chamber via the openings 406 since the maximum pressure of the chamber is equal to ambient pressure, and since the inhalation valve 132 prevents the user from exhaling into the chamber. Similarly, the pMDI 156 is open to atmosphere, or the ambient environment, and already introduces an open channel to atmosphere. Alternately, as shown in FIG. 13A and B, one-way valves 408, shown as flexible flaps, may be disposed over the acoustical openings 406. The valves 408 are openable to the interior of the chamber in order to prevent air from escaping the chamber when the user is not inhaling. The valves 408, or flaps, will open on inhalation as shown in FIG. 13B, and stay open during excessive inhalation, allowing the sound of the whistle to escape from the chamber as the flaps 408 are open. When the user is not inhaling, in particular in the scenario where the pMDI 156 is actuated before the user starts inhaling, and when the pressure inside the chamber momentarily increases due to the expansion of the aerosol plume, which may cause an momentary outflow through an open sound vents, the flaps 408 would be closed and would prevent any outflow from inside the chamber, as shown for example in FIG. 13A. Alternately, instead of acoustical openings such as cutouts or vents, very thin wall sections may be added to the adapter which would maximize the release of sound energy and to minimize any dampening. Similarly, the walls of the flow channel 404, where the whistle 160 is located, may be designed in such a way as to optimize sound propagation through the walls and to the outside, ambient environment including the use of strategically located thin wall sections. Alternately, in both cases where thin wall sections are used to optimized sound transmission, different materials, specifically chosen for their acoustic properties, may be incorporated locally where it would have the most benefit on the release of sound energy to the outside environment and not let air to flow through.

The audible flow indicator 160, or whistle, may alert the user and/or caregiver that the inhalation rate is excessive, which has a negative impact on drug deposition. A single tone produced by a whistle may not be as alarming/off-putting as two (or more) simultaneous tones which are at least one semitone apart. As such, a plurality of audible flow indicators 160, or whistles, may emit sound frequencies that are one or more semitone apart and have the same trigger pressure. The use of a plurality of different whistles may result in a more alarming sound, which may have an advantageous effect on the user and/or caregiver, so as to thereby provide a more suitable warning that will be acted on. In an alternate embodiment, shown for example in FIGS. 11B-D, the whistle 460 may have two reeds 462 arranged in various configurations. In other embodiments, the use of first and second whistles with first and second tones being different by at least one semi-tone may be incorporated into other holding chambers and adapters not having a separate channel. Shown in the images are a standard single reed whistle 160 having a single reed 462, a dual reed whistle 460 where the reeds 462 are adjacent to one another and share the same resonance channel 464 in stacked and side-by-side configurations (FIG. 11B and C), and a qual reed whistle where the reeds 462 have separate confined resonance channels 466, 468 (FIG. 11D).

In one embodiment, the inhalation valve 132 has a first flow resistance, the first flow indicator 160 has a second flow resistance, and the second flow indicator 260 has a third flow resistance, wherein the second flow resistance is greater than the first flow resistance, and the first flow resistance is greater than the third flow resistance. In this way, in one embodiment, the valved holding chamber system 100 includes a first flow path (FP1) between the input end 107 and the user interface 104, wherein the input end is configured for coupling to a medicament delivery device, or pMDI 156, and wherein the first flow path (FP1) includes an inhalation valve 132 having a first flow resistance. The second flow path (FP2) is defined between the input end 107 and the user interface 104, wherein the second flow path (FP2) bypasses the inhalation valve 132, wherein the second flow path includes a flow indicator 160 having a second flow resistance, and wherein the second flow resistance is greater than the first flow resistance.

In one embodiment, the third flow path (FP3) is defined between the input end 107 and the user interface 104, wherein the third flow path (FP3) is separate from the first and second flow paths, meaning the first, second and third flow paths are not in flow communication with each other, except at the respective inlets and outlets 152, 252, 154, 254, 272 communicating with the holding chamber 102 and the user interface 104, or in the combined channel 150 as shown in FIG. 10. The third flow path (FP3) has a second flow indicator 260 with a third flow resistance, wherein the first flow resistance is greater than the third flow resistance. In this way, the second flow indicator 260, when actuated, provides indicia that a threshold inhalation flow has been achieved, and communicates to the user 111 or care giver that a proper seal of the user interface 104, such as a mask, has been achieved together with an adequate inhalation flow. Conversely, the first flow indicator 160 is actuated when an excessive inhalation flow rate, greater than a predetermined threshold, has been achieved in the second flow path (FP2), such that the user 111 may adjust and lower the inhalation flow rate below the threshold. In this way, the first and second flow indicators 160, 260 provide the user with indicia about the upper and lower flow rate limits such that the user may adjust their inhalation accordingly.

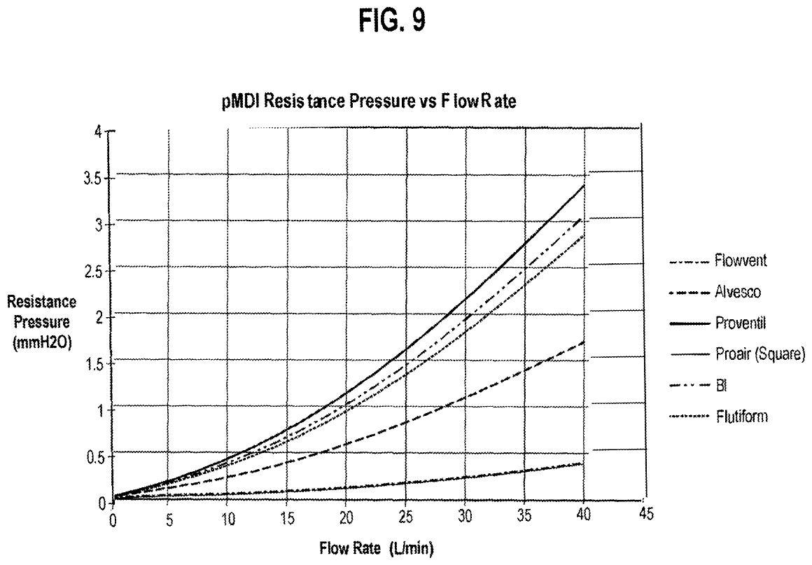

In operation, all air from the ambient environment passes through the pMDI 156 and into the holding chamber 102 before splitting, or bifurcating between the inhalation valve 132, or first flow path (FP1), and the flow indicator 150, or second flow path (FP2). The flow indicator 160 and the inhalation valve 132 each have a constant flow resistance, meaning the split of flow between the two flow paths, FP1 and FP2, will always be identical. Therefore, the flow indicator 160 may be actuated, or triggered, at a specified flow rate, regardless of the pMDI 156 in use. For example, as shown in FIG. 9, different pMDIs have different resistance pressures. In essence, the flow indicator 160 actuates independently of the pMDI 156 being used with the holding chamber 102. Upon inhalation, the air passes through the pMDI 156 and into the holding chamber 102 where it can take one of two paths-through the inhalation valve 132 (FP1), or through the flow channel 150 and through or past the flow indicator 160 (FP2). Once inside the holding chamber 102, the air will take the path of least resistance, with the majority of the airflow passing through the inhalation valve 132 and a portion passing through the flow channel(s) 150, 250. The path of least resistance is through the second flow channel 250, 404 defining the third flow path (FP3), if the system is configured with a second flow channel 250, 404. In one embodiment, the airflow reaches the flow indicator 260, or valve 262, at a trigger flow rate (between 0.5-7 lpm), thereby moving the flow indicator to indicate adequate inhalation flow. The user's inhalation flow rate is preferably between 15-30 lpm, but may be between 10 and 100 lpm. Once the trigger flow rate is achieved, the valve 262 seals against a valve seat 266, preventing further airflow through the flow channel 250, 404. With the flow channel 250, 404 closed, the airflow will pass from the pMDI 156 and through either the inhalation valve 132 or through the first flow channel 150.

Since the second flow path (FP2) has a much higher resistance than the first flow path (FP1), most of the airflow passes through the inhalation valve 132 along the first flow path (FP1), while 5-30% (10% is preferred) of the user's inhalation flow passes through the second flow path (FP2) at higher flow rates, e.g., where the inhalation flow rate exceeds 60 lpm. In this context, lower flow rates describe any flow rate less than the flow rate required to actuate the inhalation valve 132. For reference, in one embodiment, the inhalation valve 132 acts in a binary fashion, such that once the valve 132 is actuated and opened, the resistance of the first flow path (FP1) suddenly decreases. The resistance of the first flow path (FP1) is constant, given that the user's inhalation flow rate remains above the threshold for higher flow rates.

In another embodiment, shown in FIG. 2C, the flow indicator 160 may be positioned in series with the inhalation valve 132 and pMDI 156. While the flow indicator 160 functions independently of the pMDI 156, the flow indicator may be positioned in the flow path to minimize any degradation of the flow indicator or delivery of the substance. For example, the flow indicator 160 may be disposed in or on the pMDI adaptor, not in flow communication with the ambient environment, but rather in communication with any flow in the holding chamber 102. In another embodiment, the flow indicator 160, configured for example as a whistle, may be disposed in the user interface 104, for example in series with the inhalation valve 132. Again, the flow through or past the flow indicator does not originate directly from the ambient environment, but rather comes through the inhalation valve 132 from the holding chamber 102 and through the pMDI 156.

With the above description of the structure of the aerosol delivery system 100, the operation of the system 100 can be readily understood. In particular, a patient 111 engages the user interface 104 and the patient or caretaker then presses the pMDI canister 106 within the pMDI receptacle 120 of the pMDI 156 attached to the backpiece 114 located at the input end 107 of the chamber housing 108, which causes the medication to be delivered in aerosol form to the opening 112 in the manner described previously.

At or just after the time of depressing the pMDI canister 106, the patient 111 inhales. During proper inhalation, the flow indicator 260 will pivot forward in response to the inhalation pressure and flow, for example by an angle θ of between 25° to 45°, and preferably 45°, and seal against the valve seat surface 266. The angle θ may be varied to accommodate the attributes of the patient, i.e., child v. infant. Note that the visual flow indicator 260 has minimal resistance, due to its size and shape, and will respond to low tidal volumes, which is ideal for infants (tidal volume of approximately 50 cc, flow rate approximately 5 lpm) and small children (tidal volume ranging from approximately 150 to 250 cc, flow rate approximately 12 lpm). The movement of the visual flow indicator 260 against the valve seat 266 creates a seal that prevents further entrainment of air from the holding chamber 102 through the flow channel 250, 404. A user and/or caregiver who directs his or her attention to the viewing port area 280 will be able to see the movement of the flow indicator 260 as the flow indicator 260 forms the seal and so will become aware that inhalation is occurring or has occurred. Also, during inhalation, the inhalation valve 132 will open thereby allowing the aerosolized medication to exit the chamber housing 108 through the opening 112 along the first flow path (FP1) upon which the medication flows along so as to eventually be inhaled by the patient. The flow indicator 260 is positioned in parallel with the inhalation valve 132, and outside of the first flow path (FP1), or medication dispensing pathway, and thus does not compromise medication delivery.

If the inhalation flow rate is excessive, or greater than a threshold flow rate, the flow indicator 160 in the first flow channel 150, 402 will be actuated, for example by sounding an alarm such as the whistle. In response, the user may decrease the inhalation flow rate below the threshold level such that the delivery of the medication to the lungs is optimized.

Once the patient exhales or ceases to inhale, the flow indicator 260 will pivot back to its original vertical position until it engages the valve seat 268. The resiliency of the flow indicator 260 pivots or biases the indicator to the at-rest position. Again, the patient 111 or caregiver who directs his or her attention to the viewing port area 280 will be able to see the return movement of the flow indicator 260 and so will become aware that inhalation has ceased. Besides alerting the caregiver that inhalation or exhalation is occurring or has occurred, the movement of the flow indicator 260 gives the caregiver confidence that, where the patient interface 104 includes a mask for example, a proper seal is formed between the patient's face and the mask, or if a mouthpiece, that a proper seal between the use's mouth and the mouthpiece has been achieved.

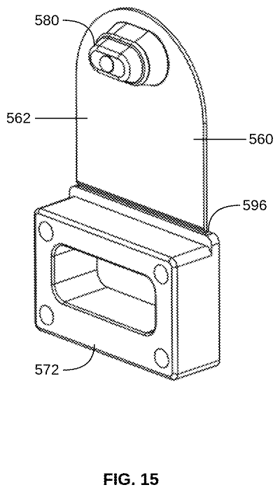

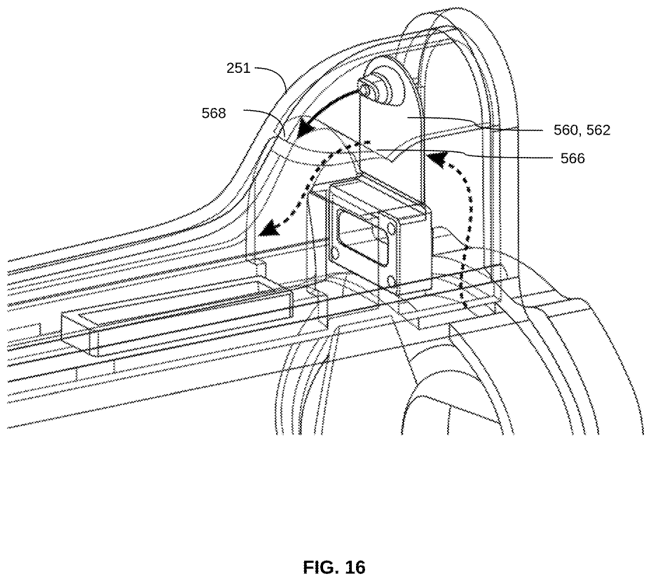

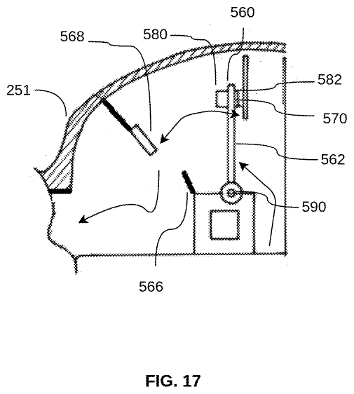

Referring to FIGS. 15-17, in situations where it is difficult to see a visual flow indicator 560, for example when a caregiver is administering medication to a child at night in a darkened room, it is preferred that the visual flow indicator 560 also make an audible noise that coincides with its movement so the same information about breath timing can be established audibly. An alternate use case is if a patient or caregiver has poor vision and has difficulty seeing the visual flow indicator 560 even with adequate lighting. An audible “tap” or similar sound is preferably created when the visual flow indicator 560 moves forward from an at-rest position to an inhalation position during inhalation and/or when it returns to the at-rest position during exhalation. In use, an audible “tap” would be heard by the caregiver on start of inhalation when the visual flow indicator 560 flap 562 pivots forward and seals against a valve seat surface 566, and with a hammer 580 engaging an impact surface or anvil 568 defined by the housing, which should be understood as any component of the system, including without limitation the holding chamber housing 108 and/or auxiliary housing 251, or any user interface connected thereto. The flap extends upwardly from a base 572, which is coupled to the housing, and may have a through-opening defining in part another flow path. In any of these systems, the visual flow indicator 560, and flap portion 562, is visible to the user and/or caregiver during inhalation. The tap of the hammer 580 striking the anvil 568 coincides with the start of inhalation. In one embodiment, the hammer 580 feature is formed of hard plastic and formed integrally with the softer, flexible, rubber visual indicator flap 562. The hammer 580 is formed near the tip of the flap 562, and is spaced apart from a pivot axis 590 of the flap, so that when the patient inhales, it moves with maximum velocity and hits the impact surface 568, or anvil, of the valve seat, which is also formed of a hard material and creates the “tap” sound as it impacts. It should be noted that advantageously, the integrally formed hard hammer 580 required to make the audible “tap” noise has an optimized amount of weight to maximize the impact force while having a minimal effect on the function of the visual indicator in response including the effect of gravity pulling on the hammer 580. Once the patient completes inhalation and begins exhaling, the visual flow indicator 560 returns to the at-rest position. The flow indicator may have a thickness of 0.1 to 2 mm. The length of the visual flow indicator is measured from the hinge area, with the sensitivity of the visual flow indicators being a function of the length of the indicator and the positioning of the hammer, or center of gravity thereof, wherein as the indicator becomes longer it becomes more sensitive to detecting flow. The visual flow indicator 560 can be integrally formed with a valve, define a valve or be made as a separate member. The indicator 560 is hingedly connected to the base 598 with a living hinge 596 formed at the junction thereof (and defining the pivot axis 590), or those components may be hingedly connected with a pin. The resiliency of the indictor flap 562 biases the indicator to an at rest position. However, it should be understood that auxiliary springs can be configured to act on the indicator to bias it to the at rest position.

In one embodiment, the hard hammer 580 is formed integrally into the flexible visual indicator flap 562 so that it protrudes from both sides and therefore defines a second hammer portion 582 similarly contacts the at-rest mating surface both at the start and end of inhalation. The hammer(s) 580, 582 may be formed as a single, homogeneous component, or may be separate components. In a similar way to inhalation, the hammer 582 contact with a hard plastic at-rest mating surface, or anvil 570, creating a similar “tap” sound which is audible to the caregiver and patient when inhalation stops and/or exhalation starts. In an embodiment where there is both a beginning of inhalation “tap” followed by an end of inhalation/beginning of exhalation “tap”, it provides the same information as the visual flow-indicator 560 but in an audible form. One “tap” corresponds with the visual flow indicator moving forward and the second “tap” corresponding to it moving back to the at rest position. In low light situations or where the visual indicator cannot be seen easily, the “tap, tap” representing the beginning and end of the inhalation can be relied upon in place of the visual aspects of the flow indicator. It can also complement the visual flow indicator even when it can easily be seen by providing a richer feedback to the caregiver or patient via visual and audible feedback complementing each other. Or for a patient who is self administering, the audible feedback can eliminate the need to look at the visual indicator or may be more effective because of the viewing angle.

It can also be appreciated that the audible form of the visual flow indicator 560 may be designed so that the audible “tap” is be generated only at the start of inhalation or only at the end of inhalation. Advantages of doing so may be to limit the number of sounds generated by the device or to clearly identify to the user that it is either the beginning or end of inhalation that is occurring but not both. For instance, drug administration is ideally performed at the start of inhalation. Therefore, only having a “tap” sound generated at the start of inhalation would provide a clear indication of that event without having to distinguish from a second “tap” sound created when inhalation has completed. This may be important when a caregiver is administering medication and cannot see the patient well to determine if they are inhaling or exhaling. In an alternate embodiment that addresses this problem, a design where a “tap” sound is created at both the start and end of inhalation is disclosed where the different “tap” sounds are unique in their audible signature. With training, the differing “tap” sounds may be associated with either start of inhalation or end of inhalation. A caregiver or patient will then know that a first “tap” sound correlates to the start inhalation and a second, different “tap” sound correlates to the end of inhalation. These sounds may also be designed to be intuitive to the user and leverage other well established sound profiles that would suggest an “in” and “out” sound profile. A higher pitch start of inhalation tap sound followed by a lower pitched tap sound for exhalation might intuitively imply in and out or the start and end of inhalation. This would be similar to the “tick”-“tock” of a clock. However, one can appreciate any number of combinations or sounds might be suitable to achieve the same result.

It should be appreciated that any number of designs could be considered to optimize the acoustics of the audible “tap” sound, whether it be generated on beginning or end of inhalation or both. In use it is important that the audible signature is both loud enough to be heard and of a pleasant and characteristic tone that is appreciated by the user or patient. In terms of loudness of the “tap” sound, one skilled in the art may appreciate that different material properties, mating part designs, and designs of the acoustic chambers in front of and behind where the sound emanates can be optimized. In various embodiments, the tap sound may be between and including 2 decibels to 10 decibels but ideally 5 decibels above ambient background noise level when measured approximately 10 cm from the distal end of the VHC. For instance, the materials selected for the impact surface, or anvils 568, 570. that the hard hammers 580, 582 impact on can advantageously be selected of materials that have a higher stiffness and hardness and therefore more likely to transmit the vibration of impact as opposed to absorbing or dampening it. A number of materials, including many thermoplastics, could be considered to meet this criteria but ideally materials that have a stiffness range of 1 GPa to 12 GPa and hardness range of Rockwell R70 to R130. The hammer(s) 580, 582, formed at the end of the visual flow indicator flap 562, may be comparatively formed of a high stiffness, high hardness, and high density material. In this aspect, ideally a thermoplastic with a minimum density of 1.5 g/cm³. Alternately, the hammer(s) may be formed of lower density materials that are suitably hard and stiff but this would require more volume of material which is less ideal in this embodiment. Alternately, the material may be a metal which would have higher densities including Aluminum (approx. 2.7 g/cm³ ) or Stainless Steel (approx. 8 g/cm³). Higher density materials allow for the maximization of the mass and therefore impact force generated for the smallest amount of volume. This is important so that the center of mass of the hammer(s) can be positioned as close as possible to the end of the visual flow indicator flap. This ensures maximum inertia with each movement and therefore maximum impact force. A material such as this is a Polyethylene terephthalate (PET)/ Polybutylene terephthalate (PBT) blend, commercially available as SABIC Valox resin, has a density of approx. 1.53 g/cm³ with a stiffness of approx. 8.5 GPa and a Hardness of approximately R118. Another aspect of the PET/PBT blend is that it is well suited to overmoulding with silicone and tolerate high temperatures required to obtain good adhesion with the silicone. This is important so that the hammer(s) maintain robust attachment to the visual flow indicator flap 562 which is ideally molded of liquid silicone rubber (LSR). However, one skilled in the art can appreciate other high stiffness, high hardness, high density materials may also be suitable.

In use, a more vigorous inhalation will generate a stronger response from the visual flow indicator flap 562 and therefore create a louder “tap” sound in comparison to a slower or less vigorous inhale. Therefore, as the patient and/or caregiver becomes familiar with the audible response, this can also provide some indication of whether the patient is inhaling too quickly or not quickly enough. For instance, if they inhale too softly to move the visual flow indicator flap sufficiently to create an audible “tap”, this may indicate to the patient or caregiver that they should inhale a little more vigorously. Conversely, if the patient inhales too vigorously, the “tap” sound may be much louder and therefore would indicate the patient or caregiver that the patient should lower their inhalation effort. The same could be applied to the embodiment described earlier which creates an audible tap on both start and end of inhalation/start of exhalation. If the user starts exhalation very vigorously, the “tap” sound generated would be louder than if they used a gentler exhalation force. In terms of the quality of the “tap” sound generated, it is desirable that the sound generated is perceived to be pleasant or positive so that this can be attributed to a positive feedback about what the patient is doing, notably that they are breathing through the device and have obtained a sufficient mouth or mask seal to support inhalation of aerosol medication.

In consideration of the audible signature of the visual flow indicator as described above, the added design features necessary to create the audible signature may also be augmented to improve the visibility of the visual flow indicator. The hammer portion(s) 580, 582, permanently attached to the end of the flexible visual flow indicator flap 562, provides a unique opportunity to make the visual flow indicator flap easier to see. Notably, the visual flow indicator flap is preferably made of a thin, rubber material which is necessary for it to be highly flexible and responsive to low inhalation flow rates. This makes it somewhat hard to see from some angles, in particular from the side. The hammer(s) 580, 582 provide added thickness, or three dimensionality, from this angle making it easier to see which may be further improved if the hammer(s) are made of a different colour, in particular a vibrant colour that would make it stand out and more visible to the user or patient. The advantage is that both improved visual and audible flow indicator performance is achieved by the addition of the hammer to the end of the flow indicator flap. In another embodiment, the materials used to form the hammer(s) 580, 582 may also be of a glow-in-the-dark nature. If the hammer(s) 580, 582 glowed in the dark, then it would augment the audible signature generated with enhanced visual feedback in low light conditions which would be able to be seen by the user or patient as the anvil moves back and forth at start and end of inhalation. It is known that the additives for plastics needed to create a robust glow in the dark response often require a high percentage loading of the material which can often impact the material properties of the base material. One skilled in the art could appreciate that while the flexible visual flow indicator flap 562 may also be pigmented with glow in the dark additives, the high loading percentage required to obtain a good glow in the dark response would have a negative impact on the desirable properties that would make it a suitable visual flow indicator flap, notably its flexibility and elasticity. Therefore, it is particularly advantageous to add glow in the dark additives to the hammer(s) which is essentially a static member that is not required to bend or flex. Only the previously mentioned desirable properties of high density, high stiffness, high hardness and ability to be overmoulded with the material used to form the visual flow indicator flap would have to be considered. However, one skilled in the art can appreciate that this can be achieved through polymer design where a suitable balance of properties can be achieved.

Alternately, while providing both visual and audible flow indicator feedback is preferrable, a design that provides only audible feedback as disclosed, where the visual portion of flow indicator flap as mentioned above is not visible. This could be because packaging or design constraints make it preferrable to integrate the flow indicator flap into the design in such a way that it allows for a more streamlined shape of the valved holding chamber. For instance, where having a protruding channel to house the flow indicator and make if visible is problematic. In this case, while the same mechanism would exist, it would be integrated into the design in such a way that it would not visible and therefore, the “tap” sound generated on either start of end of inhalation or both would be the only feedback provided.

It should be understood that the flow channels 150, 250, 402, 404, together with the first and second flow indicators 160, 260, may be incorporated into other aerosol delivery systems such as dry powder inhalers and nebulizer systems, which include chamber housings, with the flow channel.

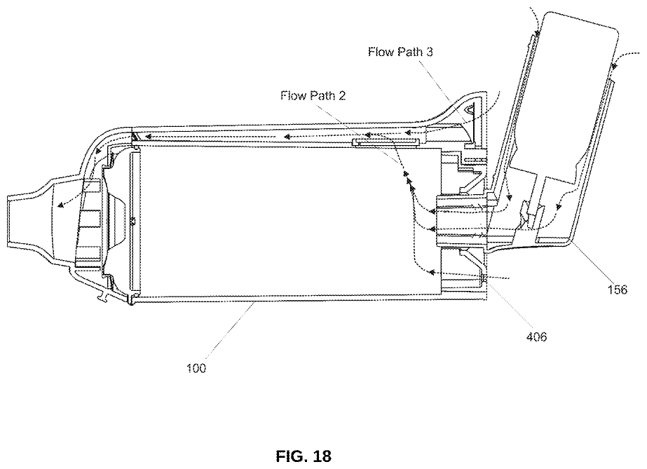

Referring to FIGS. 18-21, in an alternate embodiment, the first flow path (FP1) between the input end and the user interface is configured as in previous embodiments. A second channel inlet is located inside the chamber which forms a second flow path (FP2) through the audible flow indicator, preferably a whistle. A third air flow path (FP3) is configured to introduce ambient air into the channel via an opening in the housing directly behind the visual flow indicator 560 instead of drawing air from inside the chamber as in earlier embodiments. This approach simplifies the inlet geometry within the chamber by having one instead of two internal inlets, improves space utilization, and improves manufacturability.

In use, as the user begins to inhale through the device, air is drawn from the ambient environment through an inlet into the third flow path (FP3) and past the visual flow indicator 560, or through flow path two (FP2) via air drawn through the pMDI 156 and through the acoustical openings 406. The ratio of air flow through flow path two (FP2) and flow path three (FP3) is defined by the resistance difference between the two flow paths. In one embodiment, the resistance of flow path three (FP3) is lower than that of flow path two (FP2) such that the majority of the air is drawn through flow path three (FP3). After air has passed by the visual indicator 560 in flow path three (FP3) and past the audible indicator or whistle 460 in flow path two (FP2), the air combines in the channel 150, then flows past the channel valve, and into and out of the mouthpiece. At low inhalation flows, no air follows the first flow path (FP1) due to the fact that the primary inhalation valve 132 remains closed, as shown in FIG. 18.

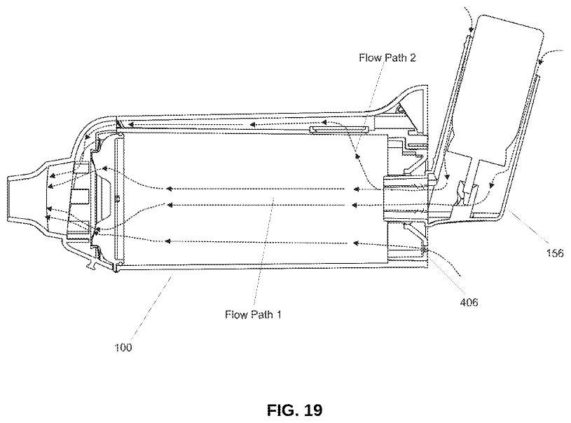

Once a threshold flow rate is reached, for example between 0.1 and 7 lpm, and preferably 3 lpm, the visual flow-indicator flap 562 moves fully forward and seals against the valve seat 566 effectively closing off the third flow path (FP3) and the source of air from ambient into the channel. This causes an increase in the resistance that initially causes the main inhalation valve 132 to open. Once the valve opens, air flow increases through the pMDI 156 and acoustic openings 406 in the backpiece 114, feeding more air into the main chamber body. With the third flow path (FP3) closed, and as defined by the resistance level of the first (FP1) and second (FP2) flow paths, air begins to split through both the first flow path (FP1) and the second flow path (FP2) through the audible flow indicator 460 and into the channel 150, as shown in FIG. 19.

The closure of the third flow path (FP3) from ambient into the channel at low flow rates, below the trigger flow rate of the audible indicator, may ensure that all air enters the audible flow indicator and the channel from inside the chamber when approaching the trigger flow rate. This maintains the configuration where variable inhalation resistance of the mating pMDI does not affect the audible flow indicator trigger flow rate.

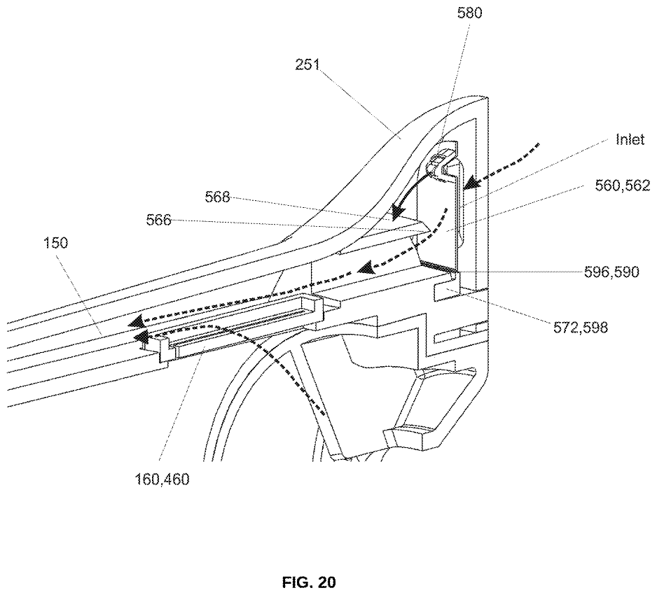

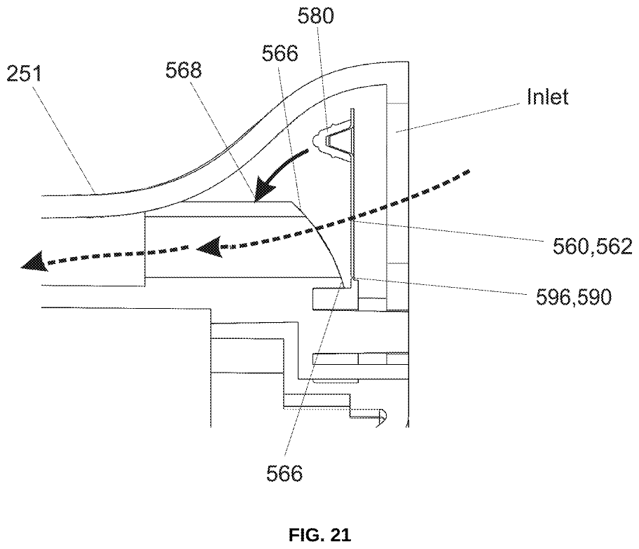

Referring to FIGS. 20 and 21, the visual flow indicator 560 in this alternate embodiment includes a flap 562 extending from a base 572, with a living hinge 596 formed at the junction thereof defining a pivot axis 590. A hammer 580 is formed near the tip of the flap 562 and is positioned to strike an anvil 568 defined by the auxiliary housing 251 when the flap 562 pivots forward during inhalation. A valve seat 566 is positioned to receive the flap 562 when the visual flow indicator moves to the inhalation position, thereby closing off the third flow path (FP3). The flow channel 150 extends from the visual flow indicator area toward the user interface, with the audible flow indicator 160, 460 positioned within the flow channel.

Referring to FIGS. 18-21, in an alternate embodiment, the first flow path (FP1) between the input end and the user interface is configured as in previous embodiments. A second channel inlet is located inside the chamber which forms a second flow path (FP2) through the audible flow indicator, preferably a whistle. A third air flow path (FP3) is configured to introduce ambient air into the channel via an opening in the housing directly behind the visual flow indicator 560 instead of drawing air from inside the chamber as in earlier embodiments. This approach simplifies the inlet geometry within the chamber by having one instead of two internal inlets, improves space utilization, and improves manufacturability.

In use, as the user begins to inhale through the device, air is drawn from the ambient environment through an inlet into the third flow path (FP3) and past the visual flow indicator 560, or through flow path two (FP2) via air drawn through the pMDI 156 and through the acoustical openings 406. The ratio of air flow through flow path two (FP2) and flow path three (FP3) is defined by the resistance difference between the two flow paths. In one embodiment, the resistance of flow path three (FP3) is lower than that of flow path two (FP2) such that the majority of the air is drawn through flow path three (FP3). After air has passed by the visual indicator 560 in flow path three (FP3) and past the audible indicator or whistle 460 in flow path two (FP2), the air combines in the channel 150, then flows past the channel valve, and into and out of the mouthpiece. At low inhalation flows, no air follows the first flow path (FP1) due to the fact that the primary inhalation valve 132 remains closed, as shown in FIG. 18.

Once a threshold flow rate is reached, for example between 0.1 and 7 lpm, and preferably 3 lpm, the visual flow-indicator flap 562 moves fully forward and seals against the valve seat 566 effectively closing off the third flow path (FP3) and the source of air from ambient into the channel. This causes an increase in the resistance that initially causes the main inhalation valve 132 to open. Once the valve opens, air flow increases through the pMDI 156 and acoustic openings 406 in the backpiece 114, feeding more air into the main chamber body. With the third flow path (FP3) closed, and as defined by the resistance level of the first (FP1) and second (FP2) flow paths, air begins to split through both the first flow path (FP1) and the second flow path (FP2) through the audible flow indicator 460 and into the channel 150, as shown in FIG. 19.

The closure of the third flow path (FP3) from ambient into the channel at low flow rates, below the trigger flow rate of the audible indicator, may ensure that all air enters the audible flow indicator and the channel from inside the chamber when approaching the trigger flow rate. This maintains the configuration where variable inhalation resistance of the mating pMDI does not affect the audible flow indicator trigger flow rate.

Referring to FIGS. 20 and 21, the visual flow indicator 560 in this alternate embodiment includes a flap 562 extending from a base 572, with a living hinge 596 formed at the junction thereof defining a pivot axis 590. A hammer 580 is formed near the tip of the flap 562 and is positioned to strike an anvil 568 defined by the auxiliary housing 251 when the flap 562 pivots forward during inhalation. A valve seat 566 is positioned to receive the flap 562 when the visual flow indicator moves to the inhalation position, thereby closing off the third flow path (FP3). The flow channel 150 extends from the visual flow indicator area toward the user interface, with the audible flow indicator 160, 460 positioned within the flow channel.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A medicament delivery system comprising:
a housing defining a flow path, wherein the housing comprises an anvil;
a flow indicator disposed across at least a portion of the flow path, wherein the flow indicator is moveable in response to an inhalation flow in the flow path from an at-rest position to an inhalation position, wherein the flow indicator comprises a hammer moveable from an at-rest position to an impact position as the flow indicator is moved in response to the inhalation flow from the at-rest position of the flow indicator to the inhalation position, wherein the hammer impacts the anvil in the impact position and emits an audible signal.

2. The medicament delivery system of claim 1 wherein the flow indicator is positioned to be visible to a user.

3. The medicament delivery system of claim 1 wherein the flow indicator comprises a flexible flap hingedly connected to the housing and defining a pivot axis, wherein the flow indicator is pivotable about the axis between the at-rest position and the inhalation position.

4. The medicament delivery system of claim 3 wherein the hammer is spaced apart from the axis.

5. The medicament delivery system of claim 4 wherein the hammer comprises a material harder than a material of the flexible flap.

6. The medicament delivery system of claim 4 wherein the hammer is made of a hard plastic.

7. The medicament delivery system of claim 1 wherein the audible signal is between and including 2 decibels and 10 decibels above an ambient background noise level when measured 10 cm from an end of the housing.

8. The medicament delivery system of claim 1 wherein the anvil has a hardness between and including Rockwell R70 to R130.

9. The medicament delivery system of claim 1 wherein the hammer has a density of at least 1.5 g/cm³.

10. The medicament delivery system of claim 1 wherein the housing defines in part a valved holding chamber having an input end and an output end.

11. The medicament delivery system of claim 1 wherein the hammer comprises a first hammer exposed on a first side of the flow indicator, and further comprising a second hammer exposed on a second side of the flow indicator opposite the first side, wherein the anvil comprises a first anvil, wherein the housing comprises a second anvil, wherein the impact position comprises a first impact position, wherein the audible signal comprises a first audible signal, and wherein the second hammer impacts the second anvil as the flow indicator is moved to the at-rest position and emits a second audible signal.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A valved holding chamber assembly comprising:
a holding chamber having an input end configured to receive a pressurized metered dose inhaler and an output end;
an inhalation valve disposed at the output end of the holding chamber and moveable to an open position in response to an inhalation flow through the holding chamber, wherein the inhalation valve defines a first inhalation flow path between the input end and a user interface;
a flow channel having an inlet in flow communication with the holding chamber between the input end and the output end and an outlet in flow communication with the user interface downstream of the inhalation valve, wherein the flow channel bypasses the inhalation valve and defines a second inhalation flow path between the input end and the user interface;
an audible flow indicator positioned in the flow channel, wherein the audible flow indicator is actuatable in response to a flow rate in the flow channel during inhalation;
a visual flow indicator defining a third inhalation flow path, wherein the third inhalation flow path includes an ambient air inlet in flow communication with an ambient environment; and
wherein the visual flow indicator is moveable from an at-rest position to an inhalation position, wherein the visual flow indicator closes the third inhalation flow path when in the inhalation position.

24. The valved holding chamber assembly of claim 23 wherein the visual flow indicator comprises a flap pivotable about a pivot axis between the at-rest position and the inhalation position.

25. The valved holding chamber assembly of claim 24 wherein the visual flow indicator further comprises a hammer positioned to strike an anvil when the visual flow indicator moves to the inhalation position.

26. The valved holding chamber assembly of claim 25 wherein the hammer emits an audible signal when striking the anvil.

27. The valved holding chamber assembly of claim 23 wherein the visual flow indicator seals against a valve seat when in the inhalation position.

28. The valved holding chamber assembly of claim 23 wherein the third inhalation flow path has a lower flow resistance than the second inhalation flow path.

29. The valved holding chamber assembly of claim 23 wherein the visual flow indicator moves to the inhalation position in response to a threshold flow rate between 0.1 and 7 lpm.

30. The valved holding chamber assembly of claim 23 wherein the audible flow indicator comprises a whistle.

31. The valved holding chamber assembly of claim 23 wherein the ambient air inlet is positioned in a housing directly behind the visual flow indicator.

32. (canceled)

33. (canceled)

34. (canceled)

35. (canceled)

36. (canceled)

37. (canceled)

38. (canceled)

39. A valved holding chamber assembly comprising:
a holding chamber having an input end and an output end;
an inhalation valve disposed at the output end defining a first flow path;
a flow channel defining a second flow path bypassing the inhalation valve, wherein the flow channel includes an audible flow indicator;
a visual flow indicator defining a third flow path, wherein the third flow path includes an inlet in direct flow communication with an ambient environment;
wherein the visual flow indicator is moveable between an at-rest position permitting airflow through the third flow path and an inhalation position closing the third flow path; and
wherein the visual flow indicator comprises a hammer configured to strike an anvil when the visual flow indicator moves to the inhalation position.

40. The valved holding chamber assembly of claim 39 wherein the third flow path has a lower flow resistance than the second flow path such that a majority of air is drawn through the third flow path when the visual flow indicator is in the at-rest position.
